(19) United States
(12) Patent Application Publication
(10) Pub. No.: US 2011/0196258 A1
(43) Pub. Date: Aug. 11, 2011

(54) NESTING ENDOSCOPIC ULTRASOUND GUIDED BIOPSY DEVICE

(76) Inventors: Shawn RYAN, Upton, MA (US); Kayla Burnim, Somerville, MA (US)

(21) Appl. No.: 13/015,794

(22) Filed: Jan. 28, 2011

Related U.S. Application Data
(60) Provisional application No. 61/301,833, filed on Feb. 5, 2010.

(51) Int. Cl. A61B 10/02

(52) U.S. Cl. 600/562

(57) ABSTRACT

A biopsy device for insertion to a target site within a living body comprises a first longitudinal element including a first longitudinal element extending from a first element proximal end to a tissue piercing first element distal end and, the first longitudinal element defining a first lumen extending therethrough from a proximal opening at the first element proximal end to a distal opening at the first element distal end. The first longitudinal element is sufficiently flexible to be inserted to a target site along a tortuous path within a natural body lumen and a second longitudinal element extending from a proximal end to a tissue piercing second element distal end. The second longitudinal element is sized to be slidably received in the first lumen and defining a second lumen extending therewithin to a tissue receiving opening at the second element distal end. The second longitudinal element is sufficiently flexible to be inserted to a target site along a tortuous path within a natural body lumen.

### PRIORITY CLAIM

This application claims the priority to the U.S. Provisional Application Ser. No. 61/301,833, entitled “NESTING ENDOSCOPIC ULTRASOUND GUIDED BIOPSY DEVICE” filed Feb. 5, 2010. The specification of the above-identified application is incorporated herewith by reference.

### BACKGROUND

Needle biopsies are common for the diagnosis and the staging of disease. In particular, in endoscopic ultrasound-guided fine needle aspiration (EUS-FNA), a biopsy needle is imaged under ultrasound so that the physician is able to visualize a position of the needle in relation to target tissue. Thus, EUS-FNA procedures ensure that the correct tissue is sampled while minimizing risk to the patient. Although EUS-FNA is a highly sensitive and specific procedure, in certain situations it may be difficult to acquire a satisfactory sample. For example, in some situations the needle must be advanced to the target site within the body along a tortuous path (e.g., through a natural body lumen) making it difficult to employ stiffer, large diameter needles. In addition, such larger gauge needles may collect tissue samples including a large amount of blood or other fluids which may interfere with analysis of the sampled tissue.

### SUMMARY OF THE INVENTION

The present invention is directed to a biopsy device for insertion to a target site within a living body, comprising a first longitudinal element including a first longitudinal element extending from a first element proximal end to a tissue piercing first element distal end and, the first longitudinal element defining a first lumen extending therethrough from a proximal opening at the first element proximal end to a distal opening at the first element distal end, the first longitudinal element being sufficiently flexible to be inserted to a target site along a tortuous path within a natural body lumen and a second longitudinal element extending from a proximal end to a tissue piercing second element distal end, the second longitudinal element being sized to be slidably received in the first lumen and defining a second lumen extending therewithin to a tissue receiving opening at the second element distal end, the second longitudinal element being sufficiently flexible to be inserted to a target site along a tortuous path within a natural body lumen.

### BRIEF DESCRIPTION OF THE DRAWINGS

FIG. 1 shows a side view of a system according to an exemplary embodiment of the present invention; and

FIG. 2 shows a side view a coupling of first and second needle devices of the system of FIG. 1;

FIG. 3 shows a cross-sectional side view of a portion of nested first and second longitudinal elements;

FIG. 4 shows a side view of a distal end of the system of FIG. 1; and

FIG. 5 shows an enlarged side view of the distal end of FIG. 4.

### DETAILED DESCRIPTION

The present invention may be further understood with reference to the following description and the appended drawings, wherein like elements are referred to with the same reference numerals. The present invention relates to endoscopic devices and, in particular, relates to endoscopic biopsy devices. An exemplary embodiment of the present invention is directed to an endoscopic biopsy device including a first needle inserted into the body to a target area and a second needle insertable through a lumen of the first needle to access the target area and collect a tissue sample therefrom. It will be understood by those of skill in the art that the nesting of the second needle within the first needle permits the target area to be sampled multiple times while minimizing trauma to the target area and to the tissue surrounding the path along which the device is inserted to the target site. It should be noted that the terms distal and proximal, as used herein, are intended to indicate a direction away from (distal) and toward (proximal) a user of the device.

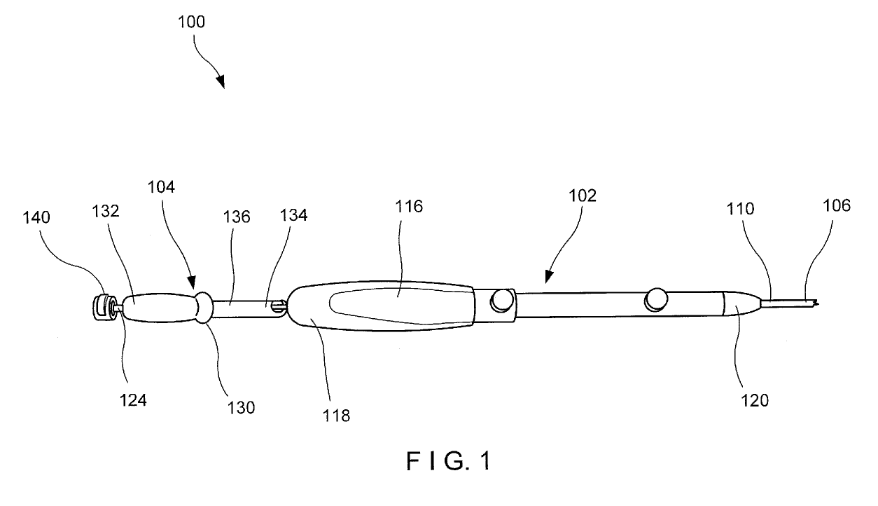

As shown in FIGS. 1-5, a system 100 according to an exemplary embodiment of the present invention comprises a first needle 102 and a second needle 104 connectable to one another. The first needle 102 includes a first longitudinal element 106 defining a lumen 114 extending therethrough from an open proximal end 110 to an open distal end 112. The first lumen 114 is sized and shaped to accommodate a second longitudinal element 108 of the second needle 104. The first longitudinal element 106 may be flexible along a length thereof such that the first longitudinal element 106 may be inserted into the body along a tortuous path (e.g., within a body lumen) to reach a target area within the body.

A proximal end 110 of the first longitudinal element 106 is coupled to a first handle 116 which extends from a proximal end 118 to a distal end 120 and includes a channel (not shown) extending therethrough sized to slidably receive the first longitudinal element 106 therethrough. The length of the first longitudinal element 106 is selected so that, when inserted through the channel of the first handle 116 with the proximal end 110 of the first longitudinal element 106 coupled thereto in a desired configuration, a distal end 112 extends distally out of the first handle 116 by a desired distance (e.g., a distance equal to a length of an endoscope to be coupled to the first handle 116 plus a desired projection of the needle distally beyond a distal end of the endoscope. The channel is arranged so that, when the first handle 116 is coupled to an endoscope in a desired configuration, the channel of the first handle 116 aligns with a working channel of the endoscope so that the first needle 102 is slid therethrough to an insertion/withdrawal position with the distal end 112 received within the distal end of the endoscope. As discussed below, after the endoscope has been advanced to a target location, the first needle 102 may be moved distally to a deployed position in which the distal end 112 extends distally out of the distal end of the endoscope (e.g., to penetrate target tissue).

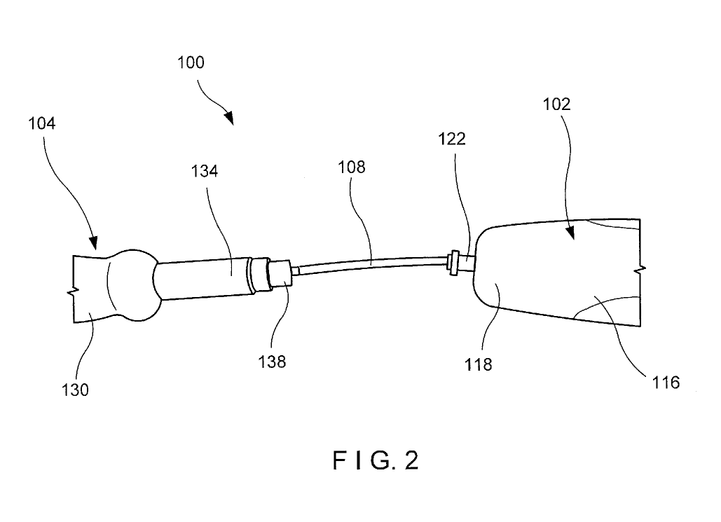

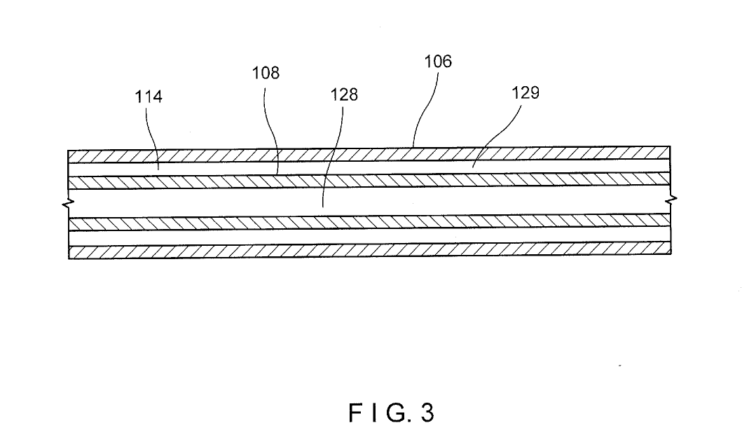

As shown in FIG. 2, the first handle 116 may further include a luer fitting 122 for coupling to the second needle 104. The second longitudinal element 108 extends longitudinally from a proximal end 124 to a distal end 126 and includes a lumen 128 extending through at least a portion of a length thereof. As shown in FIG. 3, an outer diameter of the second longitudinal element 108 is smaller than an inner diameter of the first lumen 114 of the first longitudinal element 106 such that the second longitudinal element 108 may be slidably inserted through the first lumen 114. For example, in a preferred embodiment, the first longitudinal element 106 may be a 19 gauge needle while the second longitudinal element is a 22 gauge needle. Alternatively, the first longitudinal element 106 may be a 22 gauge needle while the second longitudinal element 108 may be a 25 gauge needle.

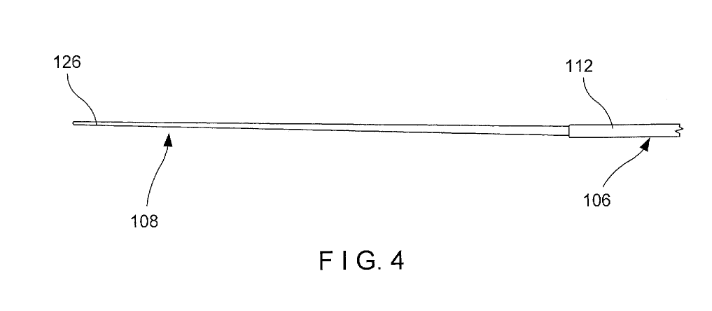

A length of the second longitudinal element 108 is preferably longer than a length of the first longitudinal element 106 such that when the second longitudinal element 108 is inserted into the first lumen 114, the distal end 126 of the second longitudinal element 108 may be extended distally past the distal end 112 of the first longitudinal element 106, as shown in FIG. 4 when desired. The proximal end 124 of the second longitudinal element 108 may include a stop 140 that extends radially outward from the second longitudinal element 108 to define a distal-most point to which the second longitudinal element 108 may be inserted into the first longitudinal element 106. A sample of target tissue may be collected within the distal end 126 of the lumen 128. However, it will be understood by those of skill in the art that the second longitudinal element 108 may include any other tissue collecting mechanism such as, for example, a lateral groove or a distal facing opening in the distal end 126.

A second handle 130 which may be attached to the proximal end 124 of the second longitudinal element 108 extends from a proximal end 132 to a distal end 134 and includes a channel 136 within which the extending therethrough. The channel 136 is sized and shaped to slidably receive the second longitudinal element 108 therethrough such that the second longitudinal element 108 may be inserted through the proximal end 132 of the handle until the distal end 126 of the second longitudinal element extends distally past the distal end 134 of the second handle 130. The stop 140 at the proximal end 124 of the second longitudinal element 108 prevents the proximal end 124 of the second longitudinal element 108 from sliding distally past the proximal end 132 of the second handle 130. The distal end 134 of the second handle 130 of this embodiment includes a coupling element 138 adapted and configured to mate with the luer fitting 122 of the first needle 102 such that the first and second needles 102, 104 may be connected to one another.

The second longitudinal element 108 of the second needle 104 may be inserted through the lumen 114 of the first longitudinal element 106 of the first needle 102 such that the second longitudinal element 108 is “nested” within the first longitudinal element 106. The second longitudinal element 108 may be slid through the lumen 114 until the coupling element 138 of the second needle 104 comes into contact with the luer fitting 122 of the first needle 102. The coupling element 138 and the luer fitting 122 mate with one another such that the first and second needles 102, 104 are coupled to one another. In a preferred embodiment, the luer fitting 122 and the coupling element 138 may be press-fit to one another. It will be understood by those of skill in the art, however, that although the exemplary embodiments describe the luer fitting 122 and the coupling element 138, the first and second needles 102, 104 may be coupled to one another using any known coupling mechanism. Once the first and second needles 102, 104 have been coupled to one another, the second longitudinal element 108 may be repeatedly used to collect tissue samples from the target area.

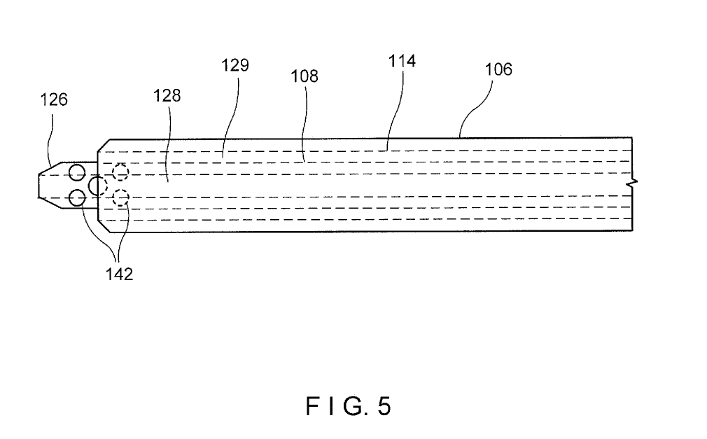

As would be understood by those skilled in the art, suction may be applied to the system 100 in an annular space 129 within the first longitudinal element 106 and outside the second longitudinal element 118 using for example, a Y-connector. In addition, as shown in FIG. 5, the system 100 may optionally include a filtering mechanism formed, for example, as a plurality of filter holes 142 at the distal end 126 of the second longitudinal element 108 sized to permit non-targeted materials such as blood and other fluids received in the lumen 128 to be suctioned through the wall of the second longitudinal element 108 into the space 129 between the first and second longitudinal elements 106, 108 while preventing target tissue received in the lumen 128 from passing therethrough. For example, where the non-targeted material is blood, each of the filter holes 142 may have a diameter of approximately 8-10 μm allowing red blood cells to flow therethrough while preventing harvested target tissue from passing out. In an alternate embodiment, each of the filter holes 142 may have a diameter in the range of 1 μm.-250 μm to conform to the requirements of a target procedure. The system 100 may further include a seal about the outer diameter of the second longitudinal element 108 at the distal end 126 that seals the space 129 to ensure that the suctioning force is transferred through the filter holes 142 to the lumen 128.

An exemplary method of use of the system 100 comprises coupling the distal end of the first handle 116 to an endoscope so that the channel extending therethrough aligns with a working channel of the endoscope. The first longitudinal element 106 of the first needle 102 is then slid through the channel of the first handle 116 into the working channel of the endoscope and advanced therethrough until the distal end 112 is in a desired position relative to the distal end of the endoscope (e.g., in the insertion/withdrawal configuration with the distal end 112 received within the distal end of the endoscope). The second needle 104 is then inserted through the second handle 130 into the first needle 102 and passed therethrough until the distal end of the second needle 104 is in a desired position within the first needle 102 (e.g., with the distal end 126 received within the first needle 102 in the insertion/withdrawal configuration). The second handle 130 is then coupled to the first needle 102. The endoscope may then be inserted to a location adjacent to target tissue to be sampled in a known manner. The user then advances the first needle 102 distally out of the endoscope (e.g., via distal advancement of the first handle 116) to a guide configuration in which the distal end 112 penetrates the target tissue. The first needle 102 is maintained in this position to provide a very accurate guide for the second needle 104 nested therein. The second needle 104 is moved to a tissue capture configuration in which it is advanced distally through the first needle 102 into the target tissue to obtain a first tissue sample. As would be understood by those skilled in the art, in the tissue capture configuration, the second needle 104 is advanced by moving the second handle 130 distally until the second handle 130 comes into contact with the first handle 116 of the first needle 102 and the coupling element 138 of the second needle 104 is coupled to the luer fitting 122 of the first needle 102. The user may then apply suction to the space 129 to draw fluids out of the sampled tissue and the second needle 104 is withdrawn from the first needle 102 so that the tissue sample may be analyzed. Specifically, after the tissue sample has been collected, the coupling element 138 may be released from the luer fitting 122 and the second needle 104 may be drawn proximally to remove the second needle 104 from the first needle 102. If it is desired to take another tissue sample, the second needle 104 is reinserted into the first needle 106 and advanced therethrough back into the target tissue mass to obtain a second sample. Suction may then be applied to the space 129 to draw blood and other fluids out of the lumen 128 via the filter holes 142. The second needle 104 may then be withdrawn from the first needle 102 so that the second tissue sample may be removed for analysis. This procedure may be repeated as often as necessary with each sample being drawn from the same single puncture site defined by the distal end 112 of the first needle 102, thereby minimizing patient trauma.

The second needle 104 may be removed from the first needle 102 by decoupling the luer fitting 122 and the coupling element 138 of the first and second needles 102, 104, respectively. When tissue collection has been completed, the first needle 102 is withdrawn proximally back into the working channel of the endoscope into the insertion/withdrawal configuration and the endoscope and first needle 102 are removed from the body in a conventional manner.

It will be apparent to those of skill in the art that various modifications and variations can be made in the structure and the methodology of the present invention, without departing from the spirit or the scope of the invention. Thus, it is intended that the present invention cover the modifications and variations of this invention provided that they come within the scope of the appended claims and their equivalents.

What is claimed is:

1. A biopsy device for insertion to a target site within a living body, comprising:
a first longitudinal element including a first longitudinal element extending from a first element proximal end to a tissue piercing first element distal end and, the first longitudinal element defining a first lumen extending therethrough from a proximal opening at the first element proximal end to a distal opening at the first element distal end, the first longitudinal element being sufficiently flexible to be inserted to a target site along a tortuous path within a natural body lumen; and
a second longitudinal element extending from a proximal end to a tissue piercing second element distal end, the second longitudinal element being sized to be slidably received in the first lumen and defining a second lumen extending therewithin to a tissue receiving opening at the second element distal end, the second longitudinal element being sufficiently flexible to be inserted to a target site along a tortuous path within a natural body lumen.

2. The device of claim 1, wherein an outer diameter of the second longitudinal element is smaller than an inner diameter of the first lumen by an amount selected to create a fluid receiving annular space therebetween when the second longitudinal element is inserted into the first lumen.

3. The device of claim 2, wherein the second longitudinal element includes a filter at a distal end thereof, the filter permitting fluids to pass therethrough from the second lumen into the fluid receiving annular space while preventing tissue received in the second lumen from passing therethrough.

4. The device of claim 3, wherein the filter comprises a plurality of holes in a distal portion of a wall of the second longitudinal element.

5. The device of claim 4, wherein the holes of the filter are between 1 μm. and 250 μm in diameter.

6. The device of claim 4, wherein the holes of the filter are between 8 μm and 10 μm in diameter.

7. The device of claim 1, further comprising:
a coupling mechanism selectively coupling the first and second longitudinal elements to one another.

8. The device of claim 7, wherein the coupling mechanism includes a first luer fitting at a proximal end of a first handle coupleable to the first longitudinal element and a complimentary luer fitting at a distal end of a second handle coupleable to the second longitudinal element.

9. The device of claim 8, wherein the first handle including a channel extending therethrough sized to slidably receive the second longitudinal element therein so that, when the first handle is coupled to the first longitudinal element, insertion of the second longitudinal element into the channel of the first handle directs the second longitudinal element into the first lumen.

10. The device of claim 9, wherein the handle of the second needle including a channel extending therethrough such that the channel slidably receives the second longitudinal element therethrough.

11. The device of claim 1, wherein the second longitudinal element is longer than the first longitudinal element such that, when inserted into the first lumen to a tissue penetrating configuration, the distal end of the second longitudinal element extends distally past the distal end of the first longitudinal element.

12. A method, comprising:
inserting a first longitudinal element along a tortuous path into a living body until a distal end thereof penetrates tissue at a target area;
inserting a second longitudinal element of through a first lumen of the first longitudinal element to penetrate tissue at the target area and receive a tissue sample in a distal end thereof;
withdrawing the second longitudinal element from the first lumen; and
after withdrawing the second longitudinal element from the first lumen, while maintaining the distal end of the first longitudinal element in the tissue at the target area, inserting one of the second longitudinal element and a third longitudinal element into the first lumen to receive a second tissue sample therein.

13. The method of claim 12, further comprising:
coupling the first and second longitudinal elements to one another via a first luer fitting at a proximal end of a first handle coupled to the first longitudinal element and a second coupling element at a distal end of a second handle of the second longitudinal element.

14. The method of claim 13, wherein the first handle includes a first channel extending therethrough sized to slidably receive the second longitudinal element, the first channel being positioned so that, when the first handle is coupled to the first longitudinal element in a desired configuration, insertion of the second longitudinal element into the first channel directs the second longitudinal element into the first lumen.

15. The method of claim 14, wherein the second handle includes a second channel extending therethrough sized to slidably receives the second longitudinal element therein.
